(19) United States
(12) Patent Application Publication
(10) Pub. No.: US 2024/0287723 A1
(43) Pub. Date: Aug. 29, 2024

(54) MEDICAL TEXTILE WASH DECONTAMINATION AND CLEANING SYSTEM

(71) Applicant: REVOLUTION-ZERO GROUP LTD, Alton (GB)

(72) Inventors: Thomas Andrew Dawson, Alton (GB); Manhar Sharma, Cambridge (GB)

(21) Appl. No.: 18/586,563

(22) Filed: Feb. 26, 2024

Related U.S. Application Data
(60) Provisional application No. 63/448,661, filed on Feb. 27, 2023.
(60) Provisional application No. 63/448,336, filed on Feb. 26, 2023.

(51) Int. Cl. D06F 34/18; D06F 33/36; D06F 34/05; D06F 34/20; D06F 103/06; D06F 103/16; D06F 103/20; D06F 103/38; G06T 7/00

(52) U.S. Cl. CPC D06F 34/18; D06F 33/36; D06F 34/05; D06F 34/20; G06T 7/001; D06F 2103/06; D06F 2103/16; D06F 2103/20; D06F 2103/38; G06T 2207/30124

(57) ABSTRACT

Validating a wash process includes establishing a surrogate reference standard by acquiring data of a candidate wash process and, additionally, including a standard cleaning marker and a standard decontamination marker; determining, through laboratory testing of the markers, that the candidate wash process was effective in cleaning and/or decontaminating the textiles washed; and, recording the acquired data and/or a function of the acquired data. A subsequent wash process then is validated by acquiring data of the subsequent wash process in the same way as in the candidate was process; recording the acquired data of the subsequent wash process in association with unique identifiers of textiles washed; and comparing the result of the function of the acquired data of the subsequent wash process with the result of the function of the acquired data of the candidate wash process, whereby the subsequent wash process is deemed effective upon a successful comparison.

### COPYRIGHT STATEMENT

Any new and original work of authorship in this document is subject to copyright protection under the copyright laws of the United States and other countries. Reproduction by anyone of this document as it appears in official governmental records is permitted, but otherwise all other copyright rights whatsoever are reserved.

### BACKGROUND

Mechanical action, chemicals, temperature, water level, water quality, and time are important variables in cleaning and decontamination of textiles by washing, especially in the context of healthcare. Standards for wash processes specifying parameters regarding one or more mechanical actions, chemicals, temperature, water level, water quality, and time have been established based on empirical data proving their effectiveness in cleaning and decontamination. A wash process that is effective is believed to be one in which, for example, the textiles are sufficiently cleaned and a quantity of infectious particles or colony forming units (CFUs) is reduced by at least several logs to an acceptably low total amount. If an instance of a wash process is determined to be compliant under an established standard or protocol, then that instance of a wash process is deemed to be effective in such cleaning and decontamination even though no testing is actually performed before or after the instance of the wash process, such as testing to determine any actual reduction in CFUs.

As an example, many standard consistent protocols specify parameters regarding exposure to a high temperature and/or to a high pH over a period of time. Such parameters might comprise a wash temperature of 75 degrees Celsius for at least 5 minutes with a pH of greater than 10 for at least 20 minutes. Data for an instance of a wash process can be tracked, i.e., monitored and recorded in a log, and then compared to the parameters of the standard for validating the instance of the wash processes. Alternatively, or in addition thereto, automated failsafe controls can be used to ensure that the standard is met. An example of such automation is a mechanism that prevents a door of a washing machine from opening until the set temperature and/or pH and time limits have been reached in accordance with the standard being applied.

The standards have worked well for conventional wash processes; however, there has been an increasing demand to alter the wash processes so as to reduce energy requirements, especially through use of reduced temperatures. The lower energy requirements of the wash processes reduce monetary costs for the energy used in heating during the wash processes and also decrease negative environmental impact through decreased CO₂ emissions. There also has been an increasing demand to use more environmentally compatible chemicals and less harsh chemicals like those that generate environments of greater than a pH of 9, even though use of environmentally friendly chemicals that can clean and disinfect typically comes at higher monetary costs. In attempting to address these increasing demands, several groups have proposed wash processes with lower wash temperatures that are claimed to be as effective at cleaning and decontaminating as wash processes performed under the established standards that utilize higher temperatures. Similarly, wash processes have been proposed that utilize more environmentally compatible chemicals that are claimed to be as effective at cleaning and decontaminating as wash processes performed under the established standards that utilize harsher chemicals with higher pH levels. Wash processes also have been proposed in which textiles are cleaned to a satisfactory level first, and then sterilized to decontaminate the textiles, and such wash processes are claimed to be as effective at cleaning and decontaminating as wash processes performed under the established standards that concurrently perform at least to some extent both cleaning and decontamination.

Unfortunately, it is challenging to confirm that the cleaning and decontamination of an instance of a wash process that does not meet an established standard nevertheless has been effective. Indeed, it is cost prohibitive to test each instance of a wash processes to confirm that the cleaning and decontamination was effective. One or more needs therefore are believed to exist for apparatus and methods for ensuring, in a manner that is operationally repeatable and cost effective, that an instance of a wash process has been effective in cleaning and decontaminating textiles. One or more embodiments in accordance with one or more aspects and features of the invention are believed to meet such needs.

### SUMMARY OF THE INVENTION

The invention includes many aspects and features. Moreover, while many aspects and features relate to, and are described in, the context of cleaning and decontamination of textiles used in healthcare, the invention is not limited to use only in such context, as will become apparent from the following summaries and detailed descriptions of aspects, features, and one or more embodiments of the invention. Thus, for example, the invention has application to and utility in the cleaning and decontamination of textiles used in cleanrooms, food processing, and the life sciences, in addition to medical services.

Generally, the invention relates to determining that one or more instances of a wash process are effective in cleaning and decontaminating textiles through laboratory testing and, thereafter, validating subsequent instances of that wash process without further testing by comparing a subsequent instance against the preceding one or more instances. The preceding one or more instances are essentially a de facto standard that is used.

In an aspect of the invention, a method for validating an instance of a wash process of textiles comprises the steps of first establishing a surrogate reference standard using a candidate instance of the wash process and, thereafter, validating a subsequent instance of the wash process using the established surrogate reference standard.

The surrogate reference standard is established by: acquiring data of the instance of the candidate wash process and, in addition thereto, including a standard cleaning marker and a standard decontamination marker with the textiles in the candidate wash process; determining, through laboratory testing of the standard cleaning marker and the standard decontamination marker, that the candidate wash process was effective in cleaning and/or decontaminating the textiles washed; and, recording in a non-transitory computer readable medium the acquired data and/or a function of the acquired data for later comparison with data acquired in the same way in a subsequent instance of the wash process whereby the effectiveness in cleaning and/or decontaminating the textiles washed in the subsequent instance can be validated without using a standard cleaning marker or a standard decontamination marker.

The subsequent instance of the wash process is validated using the surrogate reference standard that is established by: acquiring data of the instance of the wash process in the same way the acquiring data in the candidate instance was performed; recording the acquired data of the subsequent instance in a non-transitory computer readable medium in association with unique identifiers of the textiles of the instance; and comparing the instance of the wash process with the established surrogate reference standard by comparing the result of the function applied to the acquired data of the subsequent instance with the result of the function applied to the acquired data of the candidate instance based on the recording in establishing the surrogate reference standard, whereby the subsequent instance is deemed effective upon a successful comparison.

In a feature, the validating of the subsequent instance of the wash process is performed without including a standard cleaning marker or a standard decontamination marker with the textiles in the subsequent wash process.

In a feature, the acquiring of the data is performed at least in part using a reusable wash device that is included with the textiles in both the candidate instance and the subsequent instance of the wash process. The data acquired preferably comprises an identification of the washing equipment, an identification of the dosing system, an identification of the chemical containers, and identification of the drying equipment, and identification of the sterilization equipment, and identification of the operating protocols, and an identification of the reusable wash device. Furthermore, the reusable wash device preferably comprises a textile representative of the textiles of each instance of the wash process that is stained at the beginning of each instance of the wash process, and wherein the reusable wash device is configured to quantify a reduction in the stain of the representative textile resulting from the instance of the wash process.

In another aspect, a method used for validating a current instance of a wash process of textiles comprises: acquiring data of the current instance of the wash process; recording the acquired data in a non-transitory computer readable medium in association with one or more unique identifiers of the textiles of the current instance; and comparing the current instance of the wash process with a previous instance of that wash process that has been determined through testing to have been effective in cleaning and/or decontamination, wherein said comparing comprises comparing the result of a function applied to the acquired data of the current instance with the result of that function applied to data acquired in the previous instance, performance of the step of acquiring the data of the current instance being performed in the same way as data was acquired in the previous instance.

In a feature, acquiring data of the current instance of the wash process is performed at least in part using a reusable wash apparatus that is included with the textiles in the wash process.

In a feature, the acquired data is stored for online access, whereby provenance of the washed textiles may be accessed.

In a feature, a digital certificate is created regarding the validation of the current instance of the wash process and storing the digital certificate in a non-transitory computer readable medium, wherein the digital certificate comprises the acquired data of the current instance, the one or more unique identifiers of the textiles of the current instance, and a result of the comparison. The digital certificate preferably is stored for online access, whereby the provenance of a washed textile may be accessed and verified.

In a feature, acquiring data of the current instance of the wash process comprises quantifying a decrease in staining of a stained textile item that is washed with the textiles of the current instance. Quantifying a decrease in staining of the textiles of the current instance preferably comprises utilizing computer vision to inspect a textile of the current instance of the wash process at a first point in time and again at a second, subsequent point in time with at least a portion of the current instance of the wash process having been performed in the interim. This may include photographing a textile of the current instance of the wash process at a first point in time and again at a second, subsequent point in time with at least a portion of the current instance of the wash process having been performed in the interim.

In a feature, acquiring data of the current instance of the wash process comprises quantifying a decrease in staining of the textiles of the current instance. Quantifying a decrease in staining of the textiles of the current instance preferably comprises utilizing computer vision to inspect a textile of the current instance of the wash process at a first point in time and again at a second, subsequent point in time with at least a portion of the current instance of the wash process having been performed in the interim. This may include photographing a textile of the current instance of the wash process at a first point in time and again at a second, subsequent point in time with at least a portion of the current instance of the wash process having been performed in the interim.

In a feature, acquiring data of the current instance of the wash process comprises recording motions of the current instance of the washing process using a sensor apparatus that accompanies the textiles of the washing process, the sensor apparatus comprising a movement sensor.

In a feature, acquiring data of the current instance of the wash process comprises pattern matching motions of the current instance of the washing process. A pattern of a motion preferably is based on frequency, amplitude, and path of movement.

In a feature, acquiring data of the current instance of the wash process comprises digitally recording events that transpired during the wash process.

In a feature, acquiring data of the current instance of the wash process comprises acquiring data in a first manner for determining the occurrence of an event during the current instance of the wash process and further acquiring data in a second, different manner for also determining the occurrence of the same event during the current instance of the wash process, whereby the occurrence of the event is crosschecked.

In a feature, acquiring data of the current instance of the wash process comprises determining a facility at which the current instance of the wash process was performed.

In a feature of each of the foregoing methods, acquiring data of the current instance of the wash process comprises determining operators who performed one or more operations of the current instance of the wash process. This further may include identifying the wellness of each operator and, additionally, confirmation of a certification of wellness which certification is performed by a manager or supervisor of the operators.

In a feature, an RFID chip or an RFID thread comprises one the one or more unique identifiers of the textiles of the current instance. Each of the one or more unique identifiers may identify an individual textile item that is washed in the wash process, or a unique identifier may identify a bag within which textiles are washed in the wash process. The unique identifier also may simply identify a batch of textiles that are washed together in an instance of the wash process.

In a feature, a QR code or one-dimensional bar code comprises one of the unique identifiers of the textiles of the current instance.

In a feature of each of the foregoing methods, said comparing step utilizes AI/machine learning model-based analysis and/or autoencoders and/or neural networks. The AI/machine leaning and/or autoencoders may be bypassed by only comparing the time series data from the appliances and match them against the standards and, if the match crosses a threshold, the textile can be processed as passed, wherein the matching preferably is done using cross-correlation of the signals, time domain analysis and frequency domain analysis. Furthermore, the method preferably is refined over time utilizing linear and machine learning analysis.

In a feature of each of the foregoing methods, said comparing step utilizes AI/machine learning model-based analysis and/or autoencoders and/or neural networks for quantifying a decrease in staining. Furthermore, the method preferably is refined over time utilizing linear and machine learning analysis.

In another aspect, a system comprises a method that is performed according to any of the foregoing methods.

In another aspect, a reusable wash device for enabling comparison and subsequent validation of the effectiveness of a wash process comprise a processor or microcontroller, one or more sensors, and wireless receiver and transmitter, wherein the device is configured to measure and communicate data acquired during the current instant of the wash process.

In a feature, the reusable wash device comprises one or more accelerometers, one or more temperature sensors, one or more chemical sensors, one or more clocks, a non-transitory computer readable medium, a section of representative textile of the current instance, and an RFID tag, one-dimensional barcode, or QR code containing a unique identification of the device.

In a feature, data is acquired using an apparatus for photographing a textile, the apparatus comprising a compartment in which the photographs are taken, the compartment having a controlled environmental including predefined lighting and predefined distance between the camera and the representative textile.

In a feature, the device is configured to record the different devices and other components that are included in the fixed operating protocol during its encounter with each component in the wash process, including the washing equipment, drying equipment, and sterilization equipment.

In a feature, the device is configured to record the chemicals and operating protocol used during the was process.

In a feature, the device is configured to record temperature, durations of time, and/or pH readings of the wash process.

Additional aspects and features are disclosed in U.S. provisional patent applications Nos. 63/448,336 and 63/448,661 from which priority is claimed, the disclosures of which are incorporated herein by reference.

In addition to the aforementioned aspects and features of the invention, it should be noted that the invention further encompasses the various logical combinations and subcombinations of such aspects and features. Thus, for example, claims in this or a divisional or continuing patent application or applications may be separately directed to any aspect, feature, or embodiment disclosed herein, or combination thereof, without requiring any other aspect, feature, or embodiment.

### BRIEF DESCRIPTION OF THE DRAWINGS

One or more preferred embodiments of the invention now will be described in detail with reference to the accompanying drawings.

FIG. 1 is an illustration of components and steps of a method in a cleaning and decontamination validation system of an embodiment in accordance with one or more aspects and features of the invention.

FIG. 2 is an illustration of an apparatus comprising a camera and a lighting system for controlling lighting and photography conditions under which images are obtained that are representative of textiles before processing, after introduction of the standard stain, and after processing, in the decontamination and cleaning validation system of FIG. 1.

FIG. 3 illustrates representative images of photos taken using the apparatus of FIG. 2 before introduction of the standard stain (“Baseline”); after introduction of the standard stain (“After Standard Stain”), wherein the standard stain is representative as the crosshatched area in FIG. 2; and after processing (“After Processing”).

FIG. 4 illustrates a validation process of the system of FIG. 1 that utilizes four channel inputs, transformations, comparison to validated standard, and integration of results to produce a single pass-or-fail output.

FIG. 5 is an illustration representative of image-based neural network processing utilized in the system of FIG. 1.

FIG. 6 illustrates an autoencoder model that may be utilized in the system of FIG. 1.

FIG. 7 illustrates an autoencoder model with input that may be utilized in the system of FIG. 1.

FIG. 8 illustrates an area-over-and-above-the-curve analysis that may be utilized in the system of FIG. 1.

FIG. 9 illustrates a time-interval threshold analysis that may be utilized in the system of FIG. 1.

FIG. 10 illustrates a variational autoencoder that may be utilized in the system of FIG. 1.

FIG. 11 schematically illustrates a medical textile control comprising a “reusable wash device” that includes a processor or microcontroller, one or more sensors, and wireless technology (preferably Bluetooth), for measuring and communicating data taken during the process of FIG. 1, including temperature, durations (time), and/or pH readings. The sensors further may include one or more accelerometers.

FIG. 12 illustrates washing, drying, and decontamination equipment of the system of FIG. 1.

FIG. 13 illustrates a smartphone with a mobile app in the system of FIG. 1.

FIG. 14 illustrates Table 1.

FIG. 15 illustrates Table 2.

FIG. 16 illustrates Table 3.

### DETAILED DESCRIPTION

The following detailed description describes preferred apparatus, systems, and methods for validating, in a cost-effective and repeatable way, the effectiveness of cleaning and decontamination for each instance of a wash process and, preferably, medical textiles used in the provision of healthcare services.

As a preliminary matter, it will readily be understood by one having ordinary skill in the relevant art (“Ordinary Artisan”) that the invention has broad utility and application. Furthermore, any embodiment discussed and identified as being “preferred” is considered to be part of a best mode contemplated for carrying out the invention. Other embodiments also may be discussed for additional illustrative purposes in providing a full and enabling disclosure of the invention. Furthermore, an embodiment of the invention may incorporate only one or a plurality of the aspects of the invention disclosed herein; only one or a plurality of the features disclosed herein; or combination thereof. As such, many embodiments are implicitly disclosed herein and fall within the scope of what is regarded as the invention.

Accordingly, while the invention is described herein in detail in relation to one or more embodiments, it is to be understood that this disclosure is illustrative and exemplary of the invention and is made merely for the purposes of providing a full and enabling disclosure of the invention. The detailed disclosure herein of one or more embodiments is not intended, nor is to be construed, to limit the scope of patent protection afforded the invention in any claim of a patent issuing here from, which scope is to be defined by the claims and the equivalents thereof. It is not intended that the scope of patent protection afforded the invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the invention. Accordingly, it is intended that the scope of patent protection afforded the invention be defined by the issued claim(s) rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term-differs in any way from any particular dictionary definition of such term, it is intended that the meaning of the term as understood by the Ordinary Artisan should prevail.

With regard solely to construction of any claim with respect to the United States, no claim element is to be interpreted under 35 U.S.C. 112(f) unless the explicit phrase “means for” or “step for” is actually used in such claim element, whereupon this statutory provision is intended to and should apply in the interpretation of such claim element. With regard to any method claim including a condition precedent step, such method requires the condition precedent to be met and the step to be performed at least once but not necessarily every time during performance of the claimed method.

Furthermore, it is important to note that, as used herein. “comprising” is open-ended insofar as that which follows such term is not exclusive. Additionally. “a” and “an” each generally denotes “at least one” but does not exclude a plurality unless the contextual use dictates otherwise. Thus, reference to “a picnic basket having an apple” is the same as “a picnic basket comprising an apple” and “a picnic basket including an apple”, each of which identically describes “a picnic basket having at least one apple” as well as “a picnic basket having apples”; the picnic basket further may contain one or more other items beside an apple. In contrast, reference to “a picnic basket having a single apple” describes “a picnic basket having only one apple”; the picnic basket further may contain one or more other items beside an apple. In contrast, “a picnic basket consisting of an apple” has only a single item contained therein, i.e., one apple; the picnic basket contains no other item.

When used herein to join a list of items, “or” denotes “at least one of the items” but does not exclude a plurality of items of the list. Thus, reference to “a picnic basket having cheese or crackers” describes “a picnic basket having cheese without crackers”. “a picnic basket having crackers without cheese”, and “a picnic basket having both cheese and crackers”: the picnic basket further may contain one or more other items beside cheese and crackers.

When used herein to join a list of items, “and” denotes “all of the items of the list”. Thus, reference to “a picnic basket having cheese and crackers” describes “a picnic basket having cheese, wherein the picnic basket further has crackers”, as well as describes “a picnic basket having crackers, wherein the picnic basket further has cheese”; the picnic basket further may contain one or more other items beside cheese and crackers.

The phrase “at least one” followed by a list of items joined by “and” denotes an item of the list but does not require every item of the list. Thus, “at least one of an apple and an orange” encompasses the following mutually exclusive scenarios: there is an apple but no orange; there is an orange but no apple; and there is both an apple and an orange. In these scenarios if there is an apple, there may be more than one apple, and if there is an orange, there may be more than one orange. Moreover, the phrase “one or more” followed by a list of items joined by “and” is the equivalent of “at least one” followed by the list of items joined by “and”.

Additionally, as used herein “effective” and “effectiveness” in the context of a wash process refers to the degree to which textiles washed are cleaned and/or to which a quantity of infectious particles or colony forming units (CFUs) of the textiles is reduced. If the textiles are cleaned beyond a quantitative threshold and/or the reduction in the quantity of CFUs is greater than a quantitative threshold, then the cleaning and/or decontamination of the wash process is considered “effective”. As used herein, “instance” in the context of a particular wash process refers to an actual occurrence of the washing of textiles in accordance with specifications of that wash process. As used herein, “validation” and “validating” in the context of an instance of a wash process refers to the comparison of that instance of that wash process with one or more previous instances of that wash process that are known to have been effective in cleaning and/or decontamination. Thus, in validating an instance of the wash process, the textiles of that instance of a wash process are not tested to determine the effectiveness of that instance; the effectiveness of that instance is instead determined based on the comparison with the one or more previous instances of that wash process that are known through testing to have been effective. An instance of a wash process is validated if the cleaning and/or contamination of that instance is determined to have been effective based on such comparison.

Finally, as used herein “+/−” means “with in some embodiments of the invention and without in other embodiments of the invention”.

Referring now to the drawings, one or more preferred embodiments of the invention are next described. The following description of one or more preferred embodiments is merely exemplary in nature and is in no way intended to limit the invention, its implementations, or uses.

### Preferred Commercial Process Validation: The System

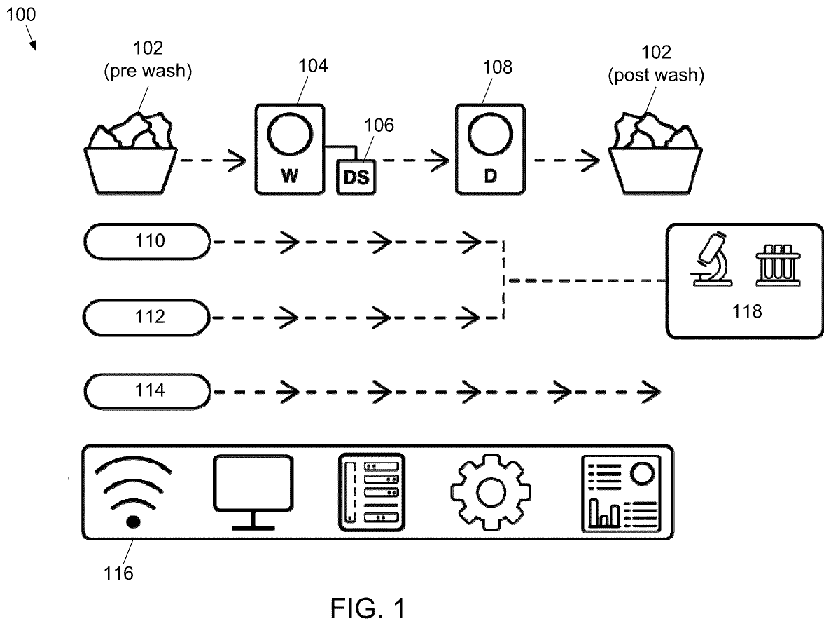

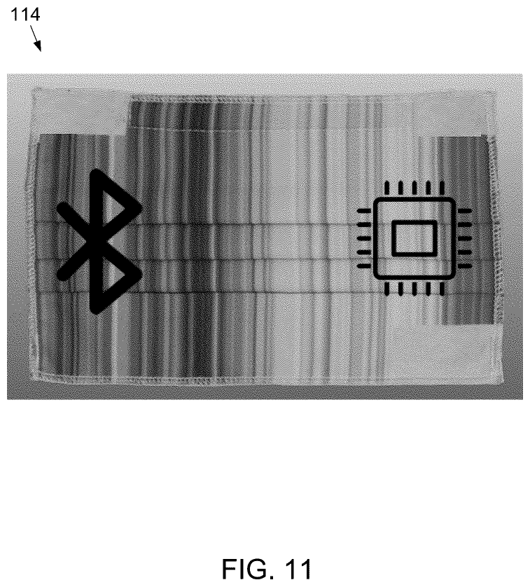

An overview of the system is illustrated in FIG. 1. The system of a preferred embodiment 100 for cleaning and decontaminating textiles 102 comprises:
Cleaning and decontamination equipment including a washer 104 and a dosing system 106, and a dryer 108, chemicals that are dispensed, operating protocols of the wash process;
Equipment and materials to test efficacy of cleaning and decontamination including standard cleaning markers 110, standard decontamination markers 112, and surrogate test markers and/or sensor systems, including the reusable wash device 114 described below and schematically illustrated in FIG. 11;
A computer software and hardware system 116 comprised of components configured for digital recording, transmission, access, storage, processing, validation and reporting;
Reference test laboratories 118 for testing standard cleaning markers and decontamination markers during initial, training, and ongoing process validation testing; and
Unique identifier technology (not shown in FIG. 1).

### Step 1

A candidate cleaning and decontamination process is selected with the specific washing/cleaning equipment, drying equipment, chemicals, textile type, textile weight loading range and fixed operating protocol defined. These variables are assigned as static absolute variables for the software validation step. Associated with these variables are static threshold variables which are static variables that may have variance associated within in pre-determined tolerances. Static absolute variables are determined by actual measurement during the process and include times, temperatures, dose amounts, and weights.

### Step 2

A water-resistant reusable (+/− wireless connected) device is provided. The “Reusable Wash Device” contains movement sensors, +/− temperature sensor, +/− chemical sensors, a clock, digital storage, a section of representative textile such as polyester, and a unique identifier such as a RFID tag, barcode or QR code. The Reusable Wash Device acts as the means for acquiring: time series measurement data from the sensors; and time series image data from the representative textile. To gain a baseline, a photo or series of photos are taken of the representative textile by a recording device of the reusable wash device.

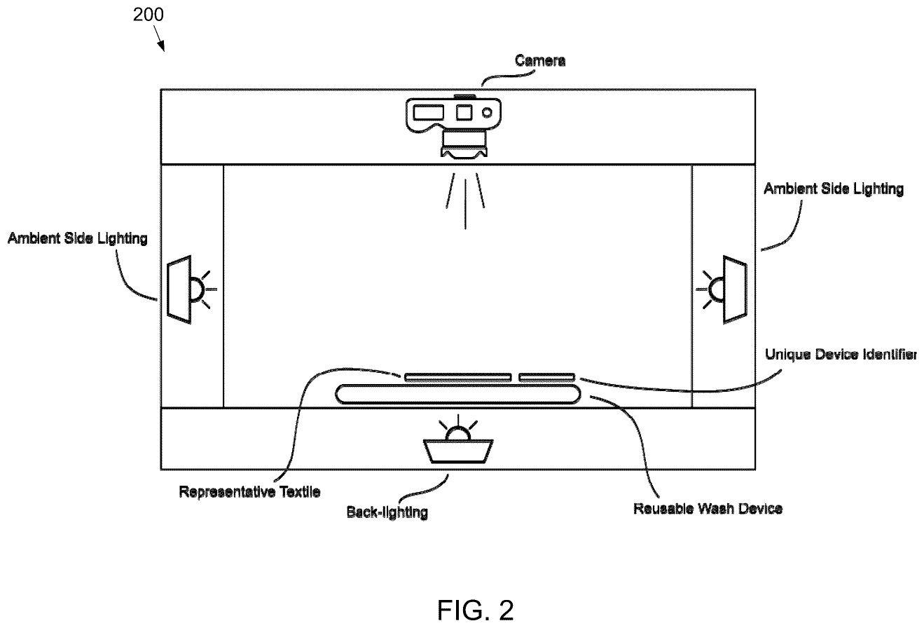

In particular, a series of photos are preferably taken. The photographs taken comprise a photo from at least one angle with +/− controlled ambient light; a photo from at least one angle with +/− controlled top lighting; a photo from at least one angle with +/− controlled backlighting; and a photo from at least one angle with +/− different hue, luminosity, and saturation settings. A combination of all could be used to optimize the analytical outputs. These photos may be taken in a specialized container 20s0 to control environmental conditions such as lighting and the distance between the camera lens and the representative textile, as shown using in FIG. 2.

The Reusable Wash Device also preferably records the different devices and other components that are included in the fixed operating protocol during its encounter with each component.

### Step 3

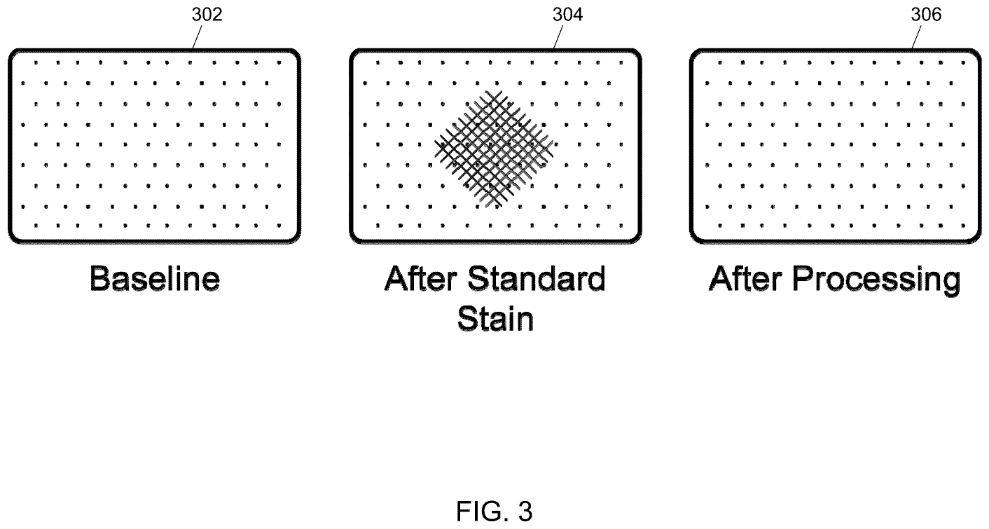

The representative textile is provided the “Standard Stain”, i.e., a standardized amount of colored compound composed of fat and protein, such as peanut butter, that is introduced to the representative textile section. A second photo or series of photos following the methods described in Step 2 of the representative textile is then also taken. FIG. 3 shows the baseline 302, the representative textile after the standard stain has been applied 304, and the representative textile after processing 306.

### Step 4

The washing equipment, drying equipment, chemicals and fixed operating protocol are logged/recorded along with the Reusable Wash Device as part of the validation system. This is logged using a digital recorder either directly and/or transcribed from manual records. The best practice step for this would by scanning and timestamping a unique identifier through utilizing a RFID tag, barcode, QR code or other unique identifier for each item being:
Washing Equipment
+/− Dosing System
Chemical Containers
Drying Equipment
+/− Sterilization Equipment
Digitally protected fixed operating protocol
Reusable Wash Device

The foregoing seven are registered with the validation system as absolute standards required for validation.

A standard wash load of 60+/−40% by weight of the washing machine capability with both a decontamination validation standard and a cleaning validation standard and the reusable wash device are then processed according to the fixed operating protocol are placed into the logged washing equipment.

To further reinforce the effectiveness of the wash process, a standard soiling load may be introduced to the textiles prior to washing at a fixed weight rate per fixed weight of textile. For example, 10 grams of 10% fat beef mince plus 10 grams of water per kilogram of textile mixed into the textile and left to soak for at least 60 minutes.

A photograph of the wash load is taken with the Reusable Wash Device (including a visual unique identifier). One of the purposes of this is for later analysis by computer vision to assess that the materials used for subsequent loads correlate with the materials that were put through the initial validation process—for example polyester, polycotton or nylon.

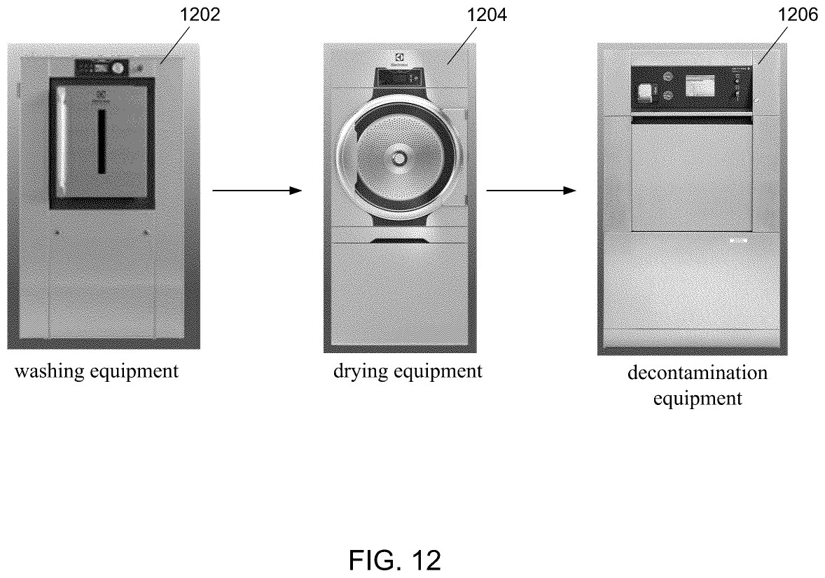

Representative washing equipment 1202, drying equipment 1204, and sterilization equipment 1206 are shown in FIG. 12.

### Step 5

The wash load completes the full candidate cleaning and decontamination process according to the fixed operating protocol. The equipment session logs from washing, drying, and sterilization equipment are registered with the digital validation system.

### Step 6

The Reusable Wash Device is removed from the processed load and a further photo or series of photos is taken of the representative textile following the methods described in Step 2 (shown in FIG. 2). These photos preferably are taken in a specialized container in which environmental conditions are controlled, such as lighting and the distance between the camera lens and the representative textile, as shown using in FIG. 2. The entire digital data associated with the fixed operating protocol session recorded on the Reusable Wash Device is uploaded to the computer system for processing, either automatically via wireless transmission, or via wired download and transfer into the system.

### Step 7

The decontamination validation standard and the cleaning validation standard are assessed utilizing a reference laboratory in consultation with relevant standards and/or regulatory authorities to determine whether the candidate cleaning and decontamination process were effective, i.e., in accordance with the established standards so as to be deemed effective.

### Step 8

If the candidate cleaning and decontamination process are deemed effective, then the total process and associated data is logged as compliant. The data log of this specifically recorded process then becomes the reference standard process for this particular fixed operating protocol, including this specific equipment.

Correlation with Standards:
Ongoing Wash to Wash that is in Compliance

### Step 9

To ensure compliance for ongoing wash loads, an analytics-derived combination and/or permutation of the compliant washing equipment, drying equipment, chemicals, operating protocols, photographic image data over time relating to the Standard Stain introduction to the representative textile, and sensor data that correlates with the validated standard process from Steps 1-8, all are required. A main difference for ongoing correlation is that there is no need to process standard cleaning markers and/or standard decontamination markers either in house or at a reference laboratory.

### Defining the Standard

That is, the combination of all the data collected during the validated cleaning and decontamination process becomes the surrogate reference standard for validating the cleaning and decontamination effectiveness of future cleaning and decontamination processes.

Analytical methods for determination that future cleaning and decontamination processes correlate and are hence compliant is achieved by crosschecking the data against binary absolutes, relative absolutes, processed image matching, and processed time series data using a combination of transformation techniques including densely connected convolutional neural networks and encoder-decoder networks.

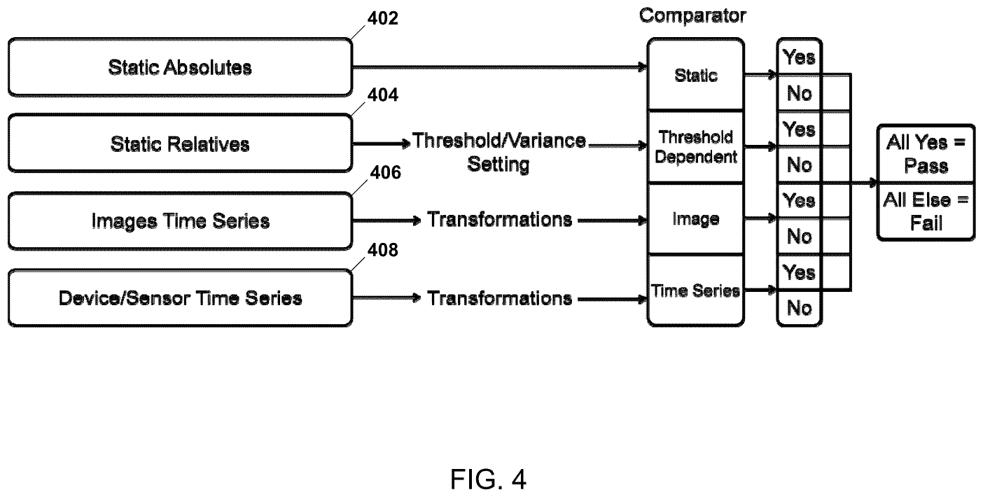

With reference to FIG. 4, the channel inputs of such processes include:

Channel #1: Static Absolutes 402, or Items 1-7, as described above. Without the confirmation of these absolutes, the cleaning and decontamination process is deemed to have failed.
Channel #2: Static Relatives 404, being static values that may have an acceptable range such as those values generated by equipment session logs, either in a raw digital or converted digital format, where measurements such as actual amount of chemical dosed, in machine temperature, or weight of wash load may vary from time to time within acceptable limits. Any recording outside those limits will result in the decontamination process being deemed to have failed.
Channel #3: Image Time Series Data 406, or a series of photographic images either in a raw digital or converted digital format, which captures the progression of the representative textile from clean to marked to processed by the introduction of the Standard Stain during Step 3 of the process described above.
Channel #4: Device Time Series Data 408 which will include the data recorded by a recording device during the fixed operating protocol. This preferably is a minimum of time series data and movement data from an accelerometer and/or gyroscope.

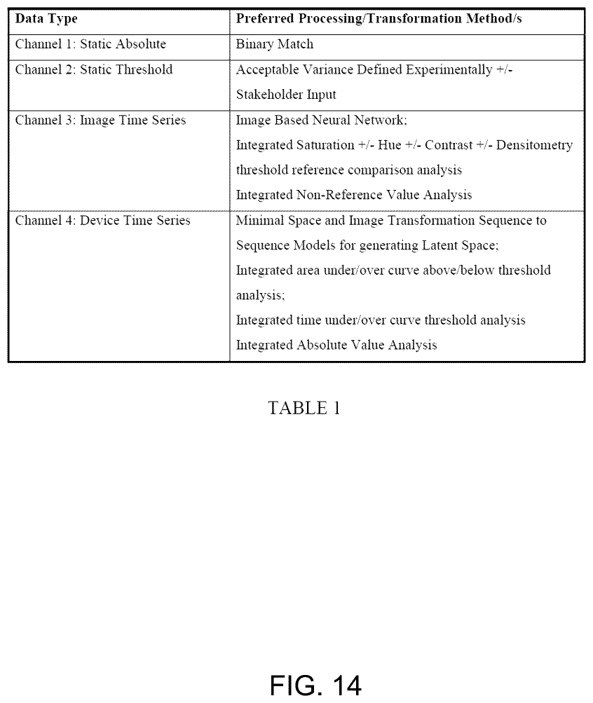

The data from channels #1 through #4 are stored on a computer system and utilized as the reference values for validation. For effective validation, the data preferably is processed as represented in Table 1 of FIG. 14. The four channel data types are processed and then together are used as matched standards using the methods as outlined below. The preferred validation is to have all four channels pass as illustrated in FIG. 4.

### Channel 1: Static Absolute

### Binary Match

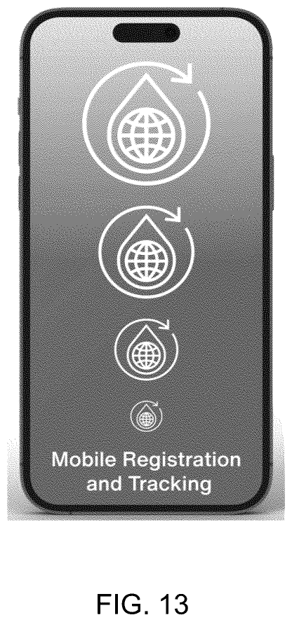

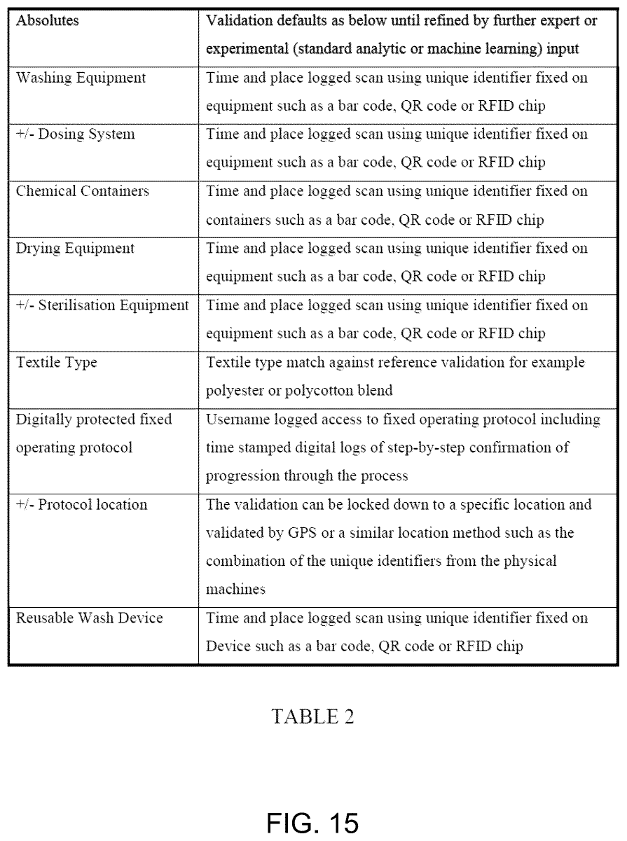

There preferably is a like-for-like match for all elements of the static absolute values. The system requires a validated and registered user to log into the recording equipment which is envisaged to be mobile recording equipment such as a portable RFID scanner or mobile phone or tablet, such as the iPhone 1300 illustrated in FIG. 13. The registered username, time, and place need to be matched with the absolutes shown in Table 2 of FIG. 15 to give a pass for Channel 1.

### Channel 2: Static Threshold

### Values +/− Variable Variance Match

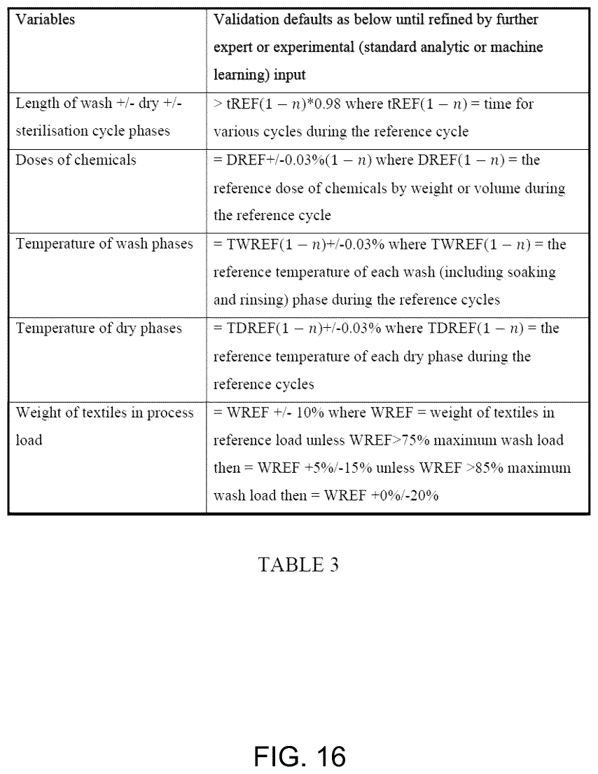

This data typically comes from process cycle logs and preferably is for a fixed time or a per weight of material processed time. The values that may have variance though would need matching within that variance are as outlined in Table 3 of FIG. 16.

### Channel 3: Image Time Series

### Method 3.1: Image Based Neural Network

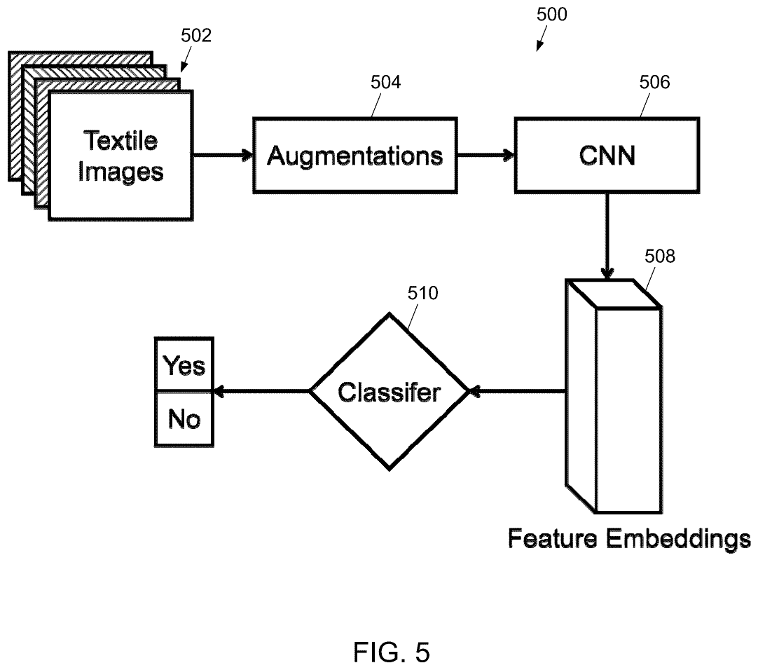

This method 500 involves an image +\− based neural network (convolutional neural network, or “CNN”). This model is preferably used to recognize useful patterns in the textile images 502 and generate feature embeddings/filters 508. In a preferred embodiment, the input textile images comprise two different images, namely, a top view image and a top view backlit image. These images are pre-processed and normalized such that, the resultant processed images follow the same distribution to make learning easier. This pre-processing is followed by data augmentations 504. The augmented images are passed through a CNN 506 to generate filters of useful features for the classifier 510. FIG. 5 shows the CNN model. The feature embeddings shown in FIG. 5 are concatenated with the embeddings learned from “Channel 4: Time Series Data validation through utilizing machine learning methods” before passing to a classifier for prediction. The data for the sequence-sequence channel 4 model preferably is generated during the cleaning cycle of the textile being validated. To validate the process has passed, this model utilizes the learned transition from pre-process image (preprocess) through to pre-process image with Standard Stain (standard stain) through the post-process image (post process). The system is trained through running a series of processes that both pass and fail the decontamination validation standard and/or the cleaning validation standard tests. Once the system is trained, this method can be used to validate the image time series channel.

### Method 3.2: Integrated Saturation+/−Hue+/−Contrast+/−Densitometry Threshold Reference Comparison Analysis

In this method the images in the image series are assessed for Saturation+/−Hue+/−Contract+/−Densitometry. The response to the Reference Cycle is calculated by taking any combination or permutation of the calculated Saturation, Hue, Contrast, Densitometry analysis of the Time Series Images and processing then as follows where:

$$Value = \sum ( Standard Stain ) - \sum ( Preprocess ) - \sum ( Postprocess )$$

Whereas the validation of the image time series channel using this method is realized if the Test Value is less than or equal to the Reference Value unless acceptable variance above Reference Value is determined by machine learning or expert regulatory input. Validated if Test Value≤ Reference Value+optional acceptable variance.

### Method 3.3: Integrated Non-Reference (Test Only) Value Analysis

This method does not rely on the Reference Test. It relies on the analysis of the Preprocess to Standard stain to Postprocess images alone where this is determined to have passed if either the:
3.3.1) Image Based Neural Network determines the differences between the Preprocess and Postprocess images are not significantly different; or
3.3.2) Integrated Saturation+/−Hue+/−Contrast+/−Densitometry Analysis where the response to the Standard stain challenge is calculated by taking any combination or permutation of the calculated Saturation, Hue, Contrast, Densitometry analysis where:

$$Value = \sum ( Preprocess ) - \sum ( Postprocess )$$

where, the validation of the time series channel using this method is realized if the Value is ≤0+optional acceptable variance as determined by machine learning or expert regulatory input.

### Channel 4: Device/Sensor Time Series

All methods below may utilize all data gathered from the Device during the whole protected protocol decontamination process. Most, however, will focus on data derived from movement during the wash cycles that occur in the washing machine. This data will typically be 3 axes or accelerometer data, 3 axes of gyroscopic data and may be supplemented by additional data including temperature and chemical sensor data such as pH, Reduction-Oxidation potential, nitrogen compounds, enzyme activity and carbonates. At a minimum there is potential for validation via the methods below utilizing just one axis of movement data over time during a wash cycle.

Any validation methods below subject refinement by further expert or experimental (standard analytic or machine learning) input.

In a further embodiment of the invention the Device Time Series data may be gathered from sensors integrated within the machines utilized during the process including the washing machine, dryer, sterilization equipment or different cleaning, decontamination and disinfection equipment.

Method 4.1: Minimal Space Sequence to Sequence Model for generating Latent Space

This method utilizes an auto-encoder which will accept a sequence of time series data. This time series data preferably is processed by an encoder to produce a latent space Z. The decoder part of the model will reconstruct the original sequence from the latent space Z.

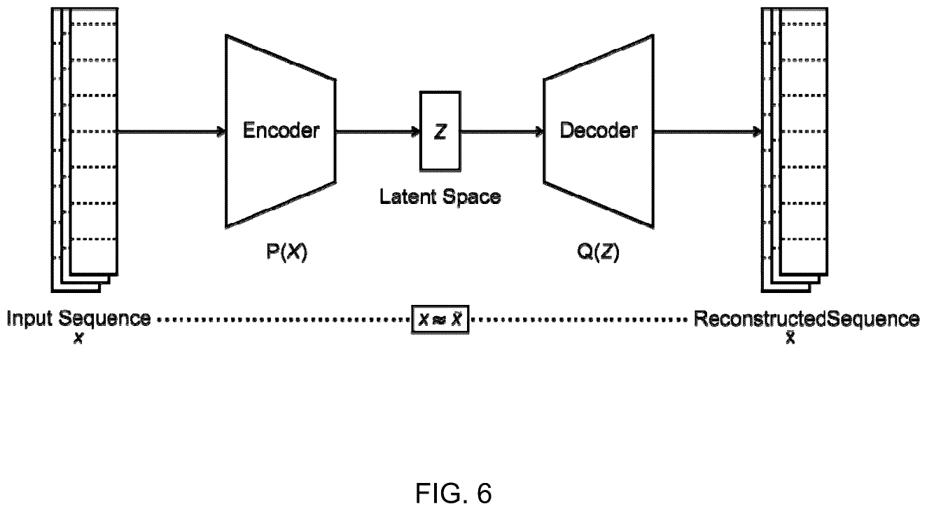

The objective is to minimize the difference between the original input X and reconstructed output data X. FIG. 6 shows two ways of achieving the said method. This latent space is a lower dimensional space of hidden representations of the original vectors in which vectors resembling each other are positioned closer to one another.

Method 4.2: Image Transformation Sequence to Sequence Model for generating Latent Space

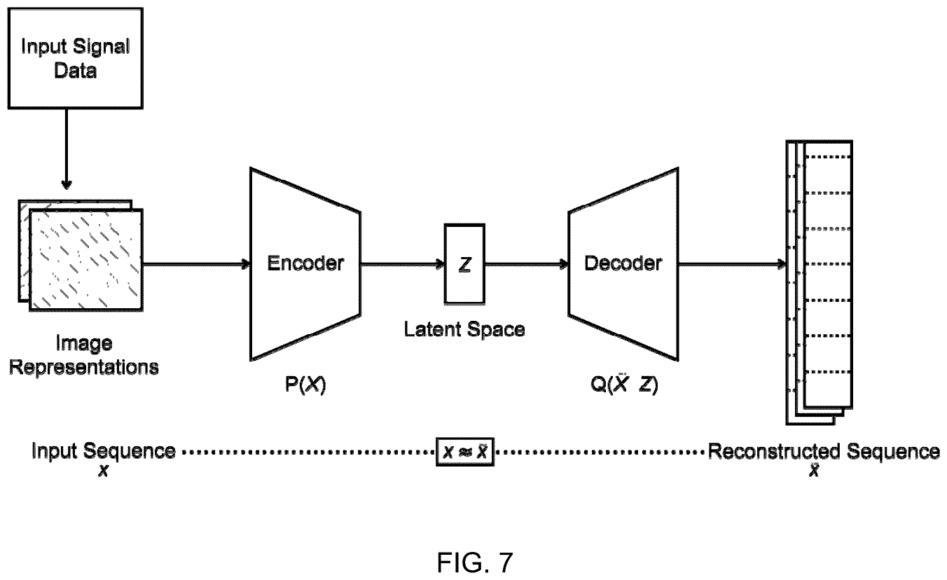

Method 4.2 transforms the time series data into image embeddings. These images can be generated by Gramian Angular Summation/Difference Fields (GASF, GADF) and/or Markov Transition Fields (MTF). To generate the image embeddings, the series data X is first rescaled between [−1,1] or [0,1] and then each point is mapped to polar coordinates. The resulting polar coordinates have the bijective property which is essential for our model. Example can be the use of arccos(xᵢ)|xᵢ∈\hat{X} where \hat{X} is the rescaled series of X. The generated images can be passed through an encoder-decoder model to learn the latent space Z as described in Method 1 and illustrated in FIG. 6. An example of the embeddings is shown in FIG. 7. This is another method for finding the embeddings for the time series data.

The goal of finding these latent space/embeddings for the time series data is to learn an efficient embedding of the input sequence (time series data) which preferably is used by a classifier along with the embeddings learned by the Convolutional neural network model in channel 3 to first make meaningful predictions. This embedding can also be used to validate Channel 4 (time series) test data as finding the closeness between the standard data (recorded using standards) embedding and test data embedding by using a similarity measure for vectors.

A mixture of both approaches 4.1 and 4.2 can be used to generate a model for learning better embeddings (Latent space) for time series data.

### Method 4.3: Integrated Area Under/Over Curve Above/Below Threshold Analysis

This method utilizes the reference model device time series data at a minimum for movement data. The movement data will typically utilize 6 data streams being 3 data streams for an accelerometer and 3 data streams from a gyroscope.

The specific data of interest that preferably is analyzed is the data derived from movement during the wash cycles that occur in the washing machine during the fixed decontamination protocol.

This method will utilize at least one movement data stream and may utilize all six plus supplementary data streams including temperature and chemical measures such as pH or Reduction-Oxidation potential.

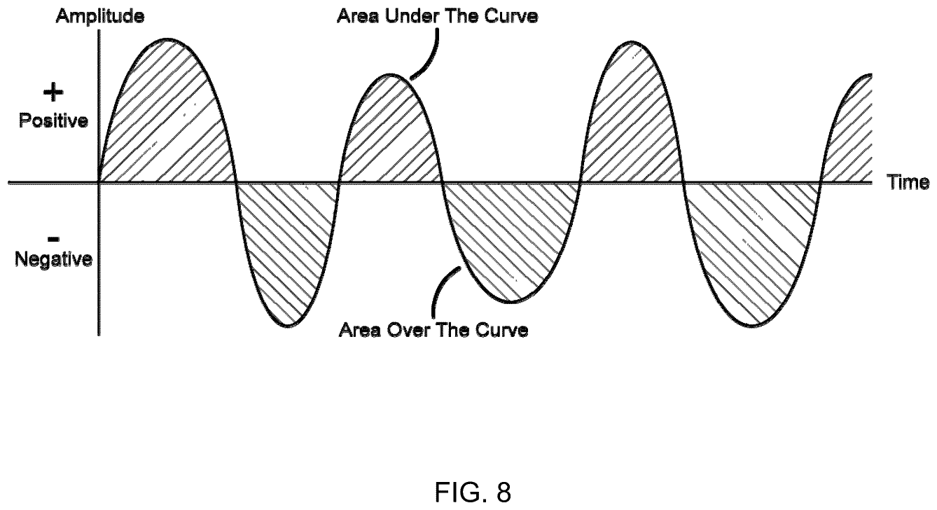

To determine the Device Time Series Validation solely utilising movement data the reference data is calculated by summing the area under or over the curve, see FIG. 8, during wash cycles=t that were recorded during the Reference Standard Process so that:

$$REFMAt = {\sum }_{1}^{n} ( absolute movement area ) sensor axes$$

$$where , REFMAt = REF ( reference ) M ( movement ) A ( area ) t ( time of wash cycle / s ) .$$

Subsequently the Test data is calculated the same way and assigned TEST(test data)MAt for matched sensor axes (1→n).

The default validation for Device Time Series utilizing this method is determined if:

$$TESTMAt ( 1 - n ) \geq REFMAt ( 1 - n )$$

This method preferably is refined by further expert or experimental (standard analytic or machine learning) input. It may utilize different thresholds on the amplitude access, different selections and numbers of axes and may also utilize inputs and different thresholds for temperature and chemical sensor data.

### Method 4.4: Integrated Time Under/Over Curve Threshold Analysis

As per Method 4.3, this method utilizes the reference model device time series data at a minimum for movement data. The movement data typically utilizes 6 data streams being 3 data streams for an accelerometer and 3 data streams from a gyroscope.

The specific data of interest that preferably is analyzed is the data derived from movement during the wash cycles that occur in the washing machine during the fixed decontamination protocol.

This method will utilize at least one movement data stream and may utilize all six plus supplementary data streams including temperature and chemical measures such as pH or Reduction-Oxidation potential.

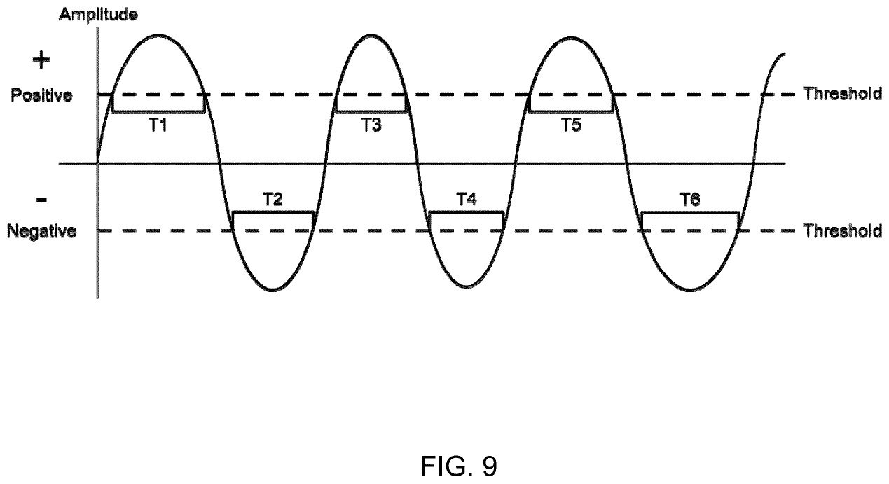

To determine the Device Time Series Validation solely utilizing movement data the reference data is calculated by time over and above specific thresholds where that threshold may be zero movement or a predetermined movement level, see FIG. 9, during wash cycles=t that were recorded during the Reference Standard Process so that

$$REFMTt = {\sum }_{1}^{n} ( absolute movement area ) sensor axes$$

$$where , REFMTt = REF ( reference ) M ( movement ) T ( time ) t ( time of wash cycle / s ) .$$

Subsequently the Test data is calculated the same way and assigned a TEST(Test Data)MTt for matched sensor axes (1→n).

A default validation for Device Time Series utilizing this method if

$$TESTMTt ( 1 - n ) \geq REFMTt ( 1 - n )$$

This method preferably is refined by further expert or experimental (standard analytic or machine learning) input. It may utilize different thresholds on the amplitude access, different selections and numbers of axes and may also utilize inputs and different thresholds for temperature and chemical sensor data.

### Method 4.5: Hybrid Methods

Combining the approaches can be utilized to generate a model for cross referencing and improving the performance of the Channel 4: Time Series Data validation through utilizing machine learning methods.

### Training and Refinement of the Validation System

The entire validation system and components of the system, including the various systems will undergo expert review, experimental validation and refinement utilizing linear and machine learning analysis. The models preferably are trained and refined through collection of experimental models to assess various thresholds for pass and failing the standard cleaning and decontamination validation tests. These may be cross referenced with emerging and more in-depth residue and microbial analysis including advanced microscopy and culturing techniques.

Utilizing these models and through the generation of representative pass and fail data based on the experimentally derived pass/fail data the entire validation system can be interrogated by altering and/or removing different validation component inputs to further test the validation and refine the analytical efficiency of the system.

Using this approach different channels can be assessed and correlated for training and refinement. For example, experimental determination to bypass the machine learning model-based validation process could be achieved by only comparing the time series data from the appliances and match them against the standards. If the match crosses a threshold, the textile can be processed as passed. This matching preferably is done using cross-correlation of the signals, time domain analysis and frequency domain analysis.

An example of this process would utilize artificially generated sensor time-series data utilizing the similar distribution patterns to the original data for the purpose of validation model training.

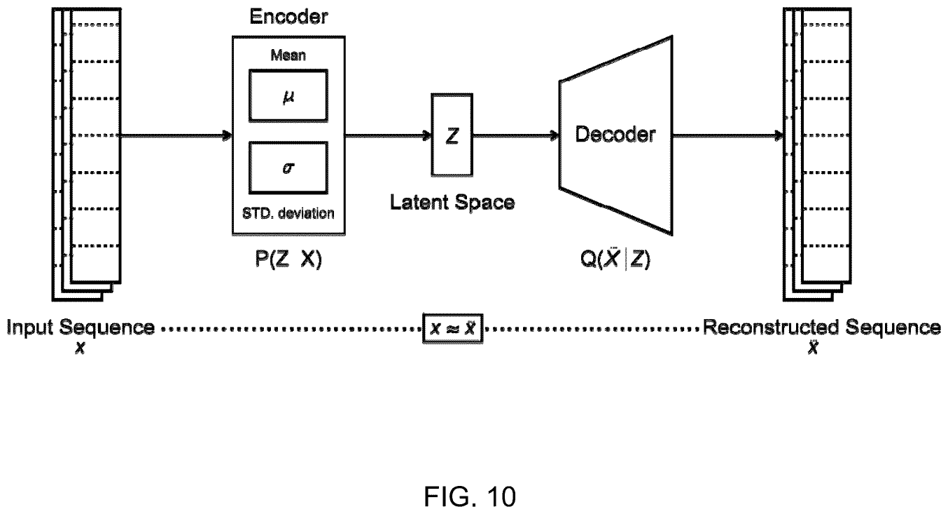

For this example, autoencoders as described in our sequence-sequence models 4.1 and 4.2 would be utilized. The change would be that the model now produces two vectors in the latent space, one for the mean of the data and one for the standard deviation of the data. This variational auto-encoder differs from the previous auto-encoder in that it forces a distribution on the latent space. It learns the distribution such that the outputs and original data have the same probability distribution. Specifically, the outputs of the model are sampled from the distribution learned by the encoder. FIG. 10 shows the variational auto-encoder.

### Periodic System Standards Compliance Testing

### Step 10

The validated standards compliance described in Steps 1-8 will need to be repeated periodically to ensure the total system remains compliant.

### Records Storage and Data Protection

All records are stored on the computer system. The records as an entirety or components of the records may be stored in a more protected format using protected data frameworks including but not limited to Blockchain, Distributed Ledger Technology, Multilayer Encryption, and Data Dispersion technologies.

### Additional Verification Through Watermarking

Additional verification of the validated technology may be required by various combinations and permutations of recorded communication between devices such as time and location stamped communication between and amongst the Reusable Wash Device, equipment and materials used during the cleaning and decontamination process, image recording equipment, computer equipment, computer software access, unique identifier read equipment, unique identifiers, other data logging equipment and human operator(s). Together as a whole or as sub-components this information can be utilized to validate the system process was correctly completed.

### Other Preferred Embodiments

It is believed that a preferred optimum cleaning and decontamination validation system comprises:
Validation of the new system which can include operating protocols, site, machines, settings and chemicals during a wash process which can include the wash only, wash and drying combined or washing and drying followed by an established sterilization method. During the entire or part of the entire process a reusable tracking device is placed in the wash load which can be comprised of sensors plus or minus markers. The validation is completed using industry standard cleaning and decontamination tests.
Once the system has been validated by the industry standards the outputs of the reusable tracking device are recorded as a “standard reusable tracking system” and as the limit for effective decontamination.
The reusable tracking system is then used for tracking and validating the effectiveness of every wash using the system with which it was originally configured. Digital assurance that this process is followed is completed by digital handshakes between the different machines and wash settings where feasible to further validate the process has been completed.
All validation points combined together into a secure digital data framework such as distributed ledger or block chain technology.
Can also include digital sign off of named assured operators and their fitness to process textiles such as digital signing off that they have had no infectious disease symptoms at the time of signed or a set time period preceding their operational duties. This sign off is also validated by a line manager or appropriate supervisor.

### Method

Part 1-Mechanical and/or Chemical Effectiveness

A device is placed in the medical textile load. The device may contain an accelerometer and/or gyroscope and/or thermometer and/or pH reader and/or may record data. This data may be wirelessly transmitted or stored locally within the device. The device may communicate with the machine that is processing the medical textile load such as a washing machine, dryer or sterilizer/autoclave. The data generated by the device could be utilized to determine whether the individual decontamination process steps, or the steps combined in various combinations were effective at cleaning and/or decontamination.

### Part 2-Cleaning Effectiveness

A mixture representative of dirt that has color associated is applied to a material that can absorb the material, coloring it. The color of this material before and after that mixture is applied is recorded via image capture. This material is then placed in a medical textile load and the color of the material may be captured during processing to determine whether or not the mixture associated with the color has been removed from the material by cleaning. Different types of image analysis including machine learning and computer vision could be utilized to determine the effectiveness of the cleaning process.

### Part 3-Cleaning Validation

Part 1 and 2 are periodically validated and/or calibrated utilising state of the art cleaning validation processes such as the EMPA technologies. The different data variables in isolation or together are utilized to qualify that Part 1 and/or Part 2 outputs correlate with effective cleaning with degree of freedom applied utilizing analytics including machine learning technologies.

### Part 4-Decontamination Validation

Part 1 and 2 are periodically validated and/or calibrated utilizing state of the art decontamination validation processes such as standard inoculated materials followed by microbiological examination.

### Controls

The best practice controls would require date, time and location logged validation, and electronic storage of that validation, that the wash process was:
Performed by a registered assured competent individual (the operator) who signs into the system and self-registers themselves as not having any infectious disease;
This wash process was additionally signed off by a competent individual in a supervisory role for the operator;
Once the operator signs into the system they log the reusable decontamination tracking device for the wash process by taking a manual or automated image of the device that is processed locally or centrally for cleaning analysis and scanning the device using optical (e.g., QR code or bar code) or electronic tagging (e.g., RFID, NFT) or manual unique identifier input;
Once the reusable decontamination tracking device is logged into the system it would ideally be in constant communication with an electronic recording and/or monitoring and/or analytics system;
The previously validated site and decontamination system equipment (e.g., dosing system, washing machine, dryer, autoclave) is also logged by scanning the site identifier or appropriate equipment using optical (e.g., QR code or bar code) or electronic tagging (e.g., RFID, NFT, Bluetooth, Wi-Fi) or manual unique identifier input;
Once the previous validated decontamination system equipment is logged into the system it would ideally be in constant communication with an electronic recording and/or monitoring and/or analytics system;
The items that are to be decontaminated are either logged individually using unique identifiers, logged together in a laundry bag, or logged together as being in the same batch that represents all of the textiles that are washed together in an instance of a wash process;
The items to be decontaminated are placed into the decontamination cycle with the reusable decontamination device logging the interaction with each decontamination equipment item which could done electronically (e.g., via Bluetooth) or manually;
If done electronically the sensor and outputs from the reusable decontamination device and decontamination equipment are analyzed by the system until they have met the requirements for decontamination of that particular phase of the decontamination cycle (for example: X Celsius wash for X minutes with the previously validated combination acceleration and G force agitation cycle/pattern over X minutes);
If the equipment has communication and integrated door control functions not allowing the cycle to finish or the door to open until it has met the requirements as per point 9;
Once the cycle with has finished the visual appearance of the reusable decontamination device is logged either automatically or through manually taking a digital photograph and either processing the photograph locally or centrally with the purpose for analysis the image for effective cleaning utilizing the change;
For each phase of the decontamination process items 9-11 are repeated until the end of the decontamination cycle;
If everything has been compliant for the whole process a digital certificate is issued that is tagged to every textile device, or the batch, that has been processed;
The digital certificate ideally contains all the information relating to the history of the textile, current and previous processing and is held within a secure data framework such as distributed ledger technology or similar;
This digital certificate is stored and can be posted to responsible stakeholders to validate that the items have been processed to the correct standards;
If they have failed, this is also logged along with the reasons for failure; and
The total system offering above can have components and subcomponents removed to improve efficiency and economy as long as it meets the minimum requirements of both legislation, regulatory authorities and responsible stakeholders.

Based on the foregoing description, it will be readily understood by those persons skilled in the art that the invention has broad utility and application. Many embodiments and adaptations of the invention other than those specifically described herein, as well as many variations, modifications, and equivalent arrangements, will be apparent from or reasonably suggested by the invention and the foregoing descriptions thereof, without departing from the substance or scope of the invention.

Accordingly, while the invention has been described herein in detail in relation to one or more preferred embodiments, it is to be understood that this disclosure is only illustrative and exemplary of the invention and is made merely for the purpose of providing a full and enabling disclosure of the invention. The foregoing disclosure is not intended to be construed to limit the invention or otherwise exclude any such other embodiments, adaptations, variations, modifications or equivalent arrangements, the invention being limited only by the claims appended hereto and the equivalents thereof.

1. A method for validating an instance of a wash process of textiles, comprising:
(a) establishing a surrogate reference standard using a candidate instance of the wash process by,
(i) acquiring data of the instance of the candidate wash process and, in addition thereto, including a standard cleaning marker and a standard decontamination marker with the textiles in the candidate wash process,
(ii) determining, through laboratory testing of the standard cleaning marker and the standard decontamination marker, that the candidate wash process was effective in cleaning and/or decontaminating the textiles washed, and
(iii) recording in a non-transitory computer readable medium the acquired data and/or a function of the acquired data for later comparison with data acquired in the same way in a subsequent instance of the wash process whereby the effectiveness in cleaning and/or decontaminating the textiles washed in the subsequent instance can be validated without using a standard cleaning marker or a standard decontamination marker; and
(b) thereafter, validating a subsequent instance of the wash process by,
(i) acquiring data of the instance of the wash process in the same way the acquiring data in the candidate instance was performed;
(ii) recording the acquired data of the subsequent instance in a non-transitory computer readable medium in association with unique identifiers of the textiles of the instance; and
(iii) comparing the instance of the wash process with the established surrogate reference standard by comparing the result of the function applied to the acquired data of the subsequent instance with the result of the function applied to the acquired data of the candidate instance based on the recording of step (a)(iv), whereby the subsequent instance is deemed effective upon a successful comparison.

2. The method of claim 1, wherein the validating of the subsequent instance of the wash process is performed without including a standard cleaning marker or a standard decontamination marker with the textiles in the subsequent wash process.

3. The method of claim 1, wherein the acquiring of the data is performed at least in part using a reusable wash device that is included with the textiles in both the candidate instance and the subsequent instance of the wash process.

4. The method of claim 3, wherein the data acquired comprises an identification of the washing equipment, an identification of the dosing system, an identification of the chemical containers, and identification of the drying equipment, and identification of the sterilization equipment, and identification of the operating protocols, and an identification of the reusable wash device.

5. The method of claim 4, wherein the reusable wash device comprises a textile representative of the textiles of each instance of the wash process that is stained at the beginning of each instance of the wash process, and wherein the reusable wash device is configured to quantify a reduction in the stain of the representative textile resulting from the instance of the wash process.

6. A method used for validating a current instance of a wash process of textiles, comprising:
(a) acquiring data of the current instance of the wash process;
(b) recording the acquired data in a non-transitory computer readable medium in association with one or more unique identifiers of the textiles of the current instance; and
(c) comparing the current instance of the wash process with a previous instance of that wash process that has been determined through testing to have been effective in cleaning and/or decontamination, wherein said comparing comprises comparing the result of a function applied to the acquired data of the current instance with the result of that function applied to data acquired in the previous instance, performance of the step of acquiring the data of the current instance being performed in the same way as data was acquired in the previous instance.

7. The method of claim 6, wherein acquiring data of the current instance of the wash process is performed at least in part using a reusable wash apparatus that is included with the textiles in the wash process.

8. (canceled)

9. The method of claim 6, further comprising creating a digital certificate regarding the validation of the current instance of the wash process and storing the digital certificate in a non-transitory computer readable medium.

10. The method of claim 9, wherein the digital certificate comprises the acquired data of the current instance, the one or more unique identifiers of the textiles of the current instance, and a result of the comparison and further comprising storing the digital certificate for online access, whereby provenance a washed textile may be accessed and verified.

11. (canceled)

12. The method of claim 6, wherein acquiring data of the current instance of the wash process comprises quantifying a decrease in staining of a stained textile item that is washed with the textiles of the current instance.

13-17. (canceled)

18. The method of claim 6, wherein acquiring data of the current instance of the wash process comprises recording motions of the current instance of the washing process using a sensor apparatus that accompanies the textiles of the washing process, the sensor apparatus comprising a movement sensor.

19. The method of claim 6, wherein acquiring data of the current instance of the wash process comprises pattern matching motions of the current instance of the washing process, wherein a pattern of a motion is based on frequency, amplitude, and path of movement.

20-21. (canceled)

22. The method of claim 6, wherein acquiring data of the current instance of the wash process comprises acquiring data in a first manner for determining the occurrence of an event during the current instance of the wash process and further acquiring data in a second, different manner for also determining the occurrence of the same event during the current instance of the wash process, whereby the occurrence of the event is crosschecked.

23. The method of claim 6, wherein acquiring data of the current instance of the wash process comprises determining a facility at which the current instance of the wash process was performed.

24. (canceled)

25. The method of claim 6, wherein an RFID chip or an RFID thread comprises one the one or more unique identifiers of the textiles of the current instance.

26-28. (canceled)

29. The method of claim 6, wherein said comparing step utilizes AI/machine learning model-based analysis and/or autoencoders and/or neural networks.

30. The method of claim 29, wherein the AI/machine leaning and/or autoencoders are bypassed by only comparing the time series data from the appliances and matching them against the standards and, if the match crosses a threshold, the textile can be processed as passed, wherein the matching preferably is done using cross-correlation of the signals, time domain analysis and frequency domain analysis.

31. The method of claim 30, further comprising refining the method utilizing linear and machine learning analysis.

32. The method of claim 6, wherein said comparing step utilizes AI/machine learning model-based analysis and/or autoencoders and/or neural networks for quantifying a decrease in staining.

33. The method of claim 32, further comprising refining the method utilizing linear and machine learning analysis.

34-40. (canceled)
